(19) United States
(12) Patent Application Publication
(10) Pub. No.: US 2024/0115766 A1
(43) Pub. Date: Apr. 11, 2024

(54) ZINC COATED IMPLANTABLE DEVICE AND METHOD OF MAKING THE SAME

(71) Applicant: DePuy Synthes Products, Inc., Raynham, MA (US)

(72) Inventors: Rajendra K. Kasinath, Warsaw, IN (US); Max A. Nye, Richfield, OH (US); Melanie T. Henault, Kent, WA (US); Ariana E. Aghevli, West Lafayette, IN (US)

(21) Appl. No.: 18/377,546

(22) Filed: Oct. 6, 2023

Related U.S. Application Data
(60) Provisional application No. 63/414,106, filed on Oct. 7, 2022.

(51) Int. Cl. A61L 27/30; A61F 2/38; A61L 27/04; A61L 27/06; A61L 27/42; A61L 27/54; A61L 27/56

(52) U.S. Cl. CPC A61L 27/306; A61F 2/38; A61L 27/045; A61L 27/06; A61L 27/427; A61L 27/54; A61L 27/56; A61F 2310/00023; A61F 2310/00029; A61F 2310/00059; A61F 2310/00598; A61L 2300/404; A61L 2400/12

(57) ABSTRACT

An orthopaedic implant having a bone-engaging layer configured to contact bone and provide antimicrobial properties and methods for making the same.

### CROSS-REFERENCE TO RELATED APPLICATIONS

This application claims the benefit of and priority to U.S. Patent Application No. 63/414,106, entitled “ZINC COATED IMPLANTABLE DEVICE AND METHOD OF MAKING THE SAME,” which was filed on Oct. 7, 2022, and which is incorporated herein by reference in its entirety.

### TECHNICAL FIELD

The present disclosure relates generally to an implantable device, and more particularly to an implantable device having antimicrobial properties.

### BACKGROUND

Joint arthroplasty is a well-known surgical procedure by which a diseased and/or damaged natural joint is replaced by a prosthetic joint. For example, in a total knee arthroplasty surgical procedure, a patient's natural knee joint is partially or totally replaced by a prosthetic knee joint or knee prosthesis. A typical knee prosthesis includes a tibial tray, a femoral component, and a polymer insert or bearing positioned between the tibial tray and the femoral component. Some knee prosthesis designs include an antimicrobial surface coating or other type of treatment on the bone-engaging surfaces of the femoral component and/or the tibial tray.

### SUMMARY

According to an aspect of the disclosure, an implantable orthopaedic component having antimicrobial properties includes a bone-engaging surface having an antimicrobial layer disposed thereon. The antimicrobial layer can include a mixture of zinc hydroxide titanate and sodium hydroxide titanate. In some embodiments, the zinc is present in an amount to provide antimicrobial properties to the orthopaedic component. The orthopaedic component may further include a bonding layer positioned between the bone-engaging surface and the antimicrobial layer.

In another illustrative aspect of the disclosure, a method of making an implantable orthopaedic component having antimicrobial properties includes contacting a bone-engaging surface of the orthopaedic component with a precursor solution to form a precursor layer. The precursor layer may then be contacted with a coating solution comprising a source of zinc to form an antimicrobial layer. The precursor layer may be configured to react with the zinc in the coating solution to undergo an ion exchange. In some embodiments, a titanium layer is deposited on the bone-engaging surface prior to contacting the bone-engaging surface with the precursor solution.

### BRIEF DESCRIPTION OF THE DRAWINGS

The detailed description particularly refers to the following figures, in which:

FIG. 1 is an exploded perspective view of an orthopaedic knee prosthesis that includes a femoral component, a tibial bearing, and a tibial tray;

FIG. 2 is a cross-sectional view of the femoral component and the tibial bearing of FIG. 1 taken along the line 2-2 of FIG. 1, as viewed in the direction of the arrows;

FIG. 3 is an enlarged cross-sectional view showing an antimicrobial layer formed on the femoral component of FIG. 1;

FIG. 4 is an enlarged cross-sectional view similar to FIG. 3, but showing the antimicrobial layer formed on the femoral component along with a bonding layer;

FIG. 5 is a diagram showing the efficacy of titanate surface modification with zinc according to an antimicrobial assay as described in Example 5;

FIG. 6 is a diagram showing the efficacy of treated acetabular cups to reduce planktonic Staph aureus in media after 24 hours as described in Example 4;

FIG. 7 is a diagram showing the efficacy of treated acetabular cups to reduce Staph aureus on the surface of cups after 24 hours as described in Example 5;

FIG. 8 is cross-sectional view of a hip prosthesis, note the femoral stem of the hip prosthesis is not shown in cross section for clarity of description; and

FIG. 9 is a perspective view of a glenoid component of a shoulder prosthesis; and.

FIG. 10 is a diagram showing the efficacy of treated tibial trays and coupons to reduce E. coli on their surface after 24 hours as described in Example 6 (from top to bottom: Control samples media, Control samples surface, Zinc treated tibial tray media, Zinc treated tibial tray surface, Zinc treated coupon media, Zinc treated coupon surface).

### DETAILED DESCRIPTION

While the concepts of the present disclosure are susceptible to various modifications and alternative forms, specific exemplary embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention as defined by the appended claims.

Terms representing anatomical references, such as anterior, posterior, medial, lateral, superior, inferior, etcetera, may be used throughout the specification in reference to the orthopaedic implants or orthopaedic prostheses described herein as well as in reference to the patient's natural anatomy. Such terms have well-understood meanings in both the study of anatomy and the field of orthopaedics. Use of such anatomical reference terms in the written description and claims is intended to be consistent with their well-understood meanings unless noted otherwise.

Various orthopaedic components can comprise a bone-engaging surface and/or comprise an antimicrobial layer disposed thereon according to the present disclosure. In an embodiment, the orthopaedic component comprises a tibial tray. In an embodiment, the orthopaedic component comprises a screw. In an embodiment, the orthopaedic component comprises a plate. In an embodiment, the orthopaedic component comprises a trauma hardware component, for instance a titanium based trauma hardware component.

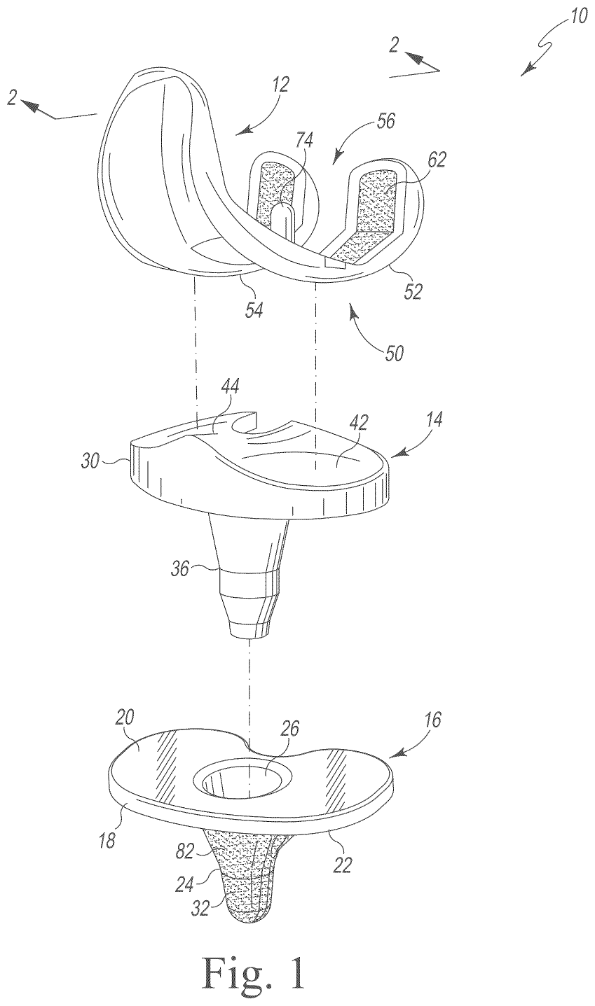

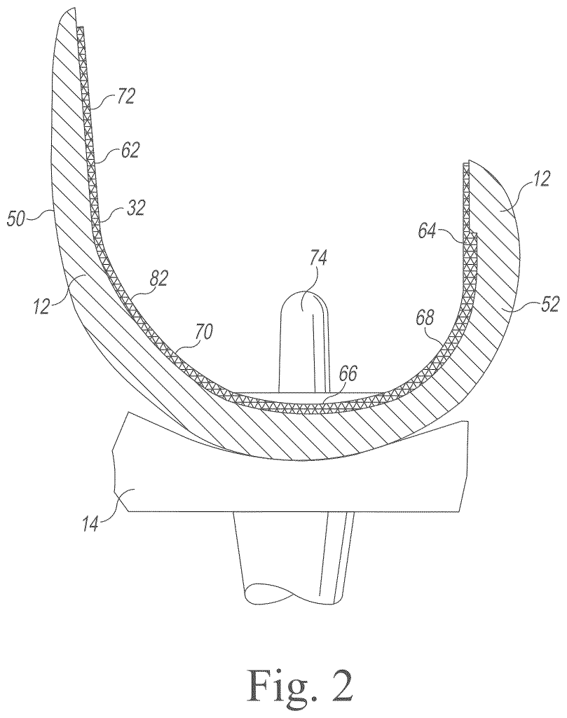

Referring to FIGS. 1 and 2, there is shown an orthopaedic knee prosthesis 10 that includes a femoral component 12, a tibial bearing 14, and a tibial tray 16. The femoral component 12 is configured to articulate with the tibial bearing 14, which is configured to be coupled with the tibial tray 16. In the illustrative embodiment of FIG. 1, the tibial bearing 14 is embodied as a rotating or mobile tibial bearing and it is, therefore, rotatable relative to the tibial tray 16. However, in other embodiments, the tibial bearing 14 may be embodied as a fixed tibial bearing (not shown), which is restricted from rotating relative to the tibial tray 16.

The tibial tray 16 is configured to be secured to a surgically-prepared proximal end of a patient's tibia (not shown). The tibial tray 16 may be secured to the patient's tibia with or without the use of bone cement. The tibial tray 16 includes a platform 18 having a superior surface 20 and an opposite inferior surface 22. The tibial tray 16 also includes a stem 24 extending downwardly from the inferior surface 22 of the platform 18. A bore 26 is defined in the superior surface 20 of the platform 18 and extends inferiorly into the stem 24. The bore 26 is configured to receive a complimentary stem 36 of the tibial bearing 14 as discussed in more detail below.

The inferior surface 22 of the platform 18 and the stem 24 define a bone-engaging surface 28 of the tibial tray 16. As can be seen in FIG. 1, the bone-engaging surface 28 has a porous-metal coating 32 disposed thereon. It should be appreciated that the porous-metal coating 32 could be a separately-applied coating such as Porocoat® Porous Coating which is commercially available from DePuy Synthes of Warsaw, Indiana. Alternatively, the porous-metal coating 32 is disposed on the metallic body 34 of the tibial tray 16 by virtue of being additively manufactured contemporaneously with the tray's metallic body 34 so as to create a common, monolithic component of the two metal structures.

As discussed above, the tibial bearing 14 is configured to be coupled with the tibial tray 16. The tibial bearing 14 includes a platform 30 having an upper bearing surface and a bottom bearing surface. In the illustrative embodiment in which the tibial bearing 14 is embodied as a rotating or mobile tibial bearing, the bearing 14 includes a stem 36 extending downwardly from the bottom surface of the platform 30. When the tibial bearing 14 is coupled to the tibial tray 16, the stem 36 is received in the bore 26 of the tibial tray 16. In use, the tibial bearing 14 is configured to rotate about an axis defined by the stem 36 relative to the tibial tray 16. In embodiments in which the tibial bearing 14 is embodied as a fixed tibial bearing, the bearing 14 may or may not include the stem 36 and/or may include other devices or features to secure the tibial bearing 14 to the tibial tray 16 in a non-rotating configuration. The upper bearing surface of the tibial bearing 14 includes a medial bearing surface 42 and a lateral bearing surface 44. The medial and lateral bearing surfaces 42, 44 are configured to receive or otherwise contact corresponding medial and lateral condyles 52, 54 of the femoral component 12. As such, each of the bearing surfaces 42, 44 has a concave contour.

Referring to FIG. 2, the femoral component 12 is configured to be coupled to a surgically-prepared surface of the distal end of a patient's femur (not shown). The femoral component 12 illustrated in FIGS. 1 and 2 is a posterior cruciate-retaining knee prosthesis and the tibial bearing 14 is embodied as a posterior cruciate-retaining tibial bearing 14. However, in other embodiments, the orthopaedic knee prosthesis 10 may be embodied as a posterior cruciate-sacrificing knee prosthesis (not shown).

As mentioned above, the femoral component 12 includes a pair of medial and lateral condyles 52, 54. The condyles 52, 54 are spaced apart to define an intracondyle notch 56 therebetween. In use, the condyles 52, 54 replace the natural condyles of the patient's femur. Each condyle 52, 54 of the femoral component 12 includes an outer articular surface 50, which is convexly curved in the sagittal plane and configured to face the respective bearing surface 42, 44 of the platform 30 of the tibial bearing 14.

Opposite to the articular surface 50, the femoral component 12 includes a bone-engaging surface 62. The bone-engaging surface 62 is the side of the femoral component that contacts the surgically-prepared distal femur of the patient. The bone-engaging surface 62 includes multiple surfaces that mate with planar surfaces surgically cut into the patient's distal femur. For example, as shown in FIG. 2, a pair of posterior fixation surfaces 64 are opposite the posterior surfaces of the condyles 52, 54, with one of the posterior fixation surfaces 64 being the medial fixation surface, the other the lateral fixation surface. As can be seen in FIGS. 1 and 2, the posterior fixation surfaces 64 extend generally in the superior/inferior direction. A pair of distal fixation surfaces 66 (one being medially positioned, the other the laterally positioned) is opposite the distal surfaces of the condyles 52, 54 and extend generally in the anterior/posterior direction. A pair of articular fixation surfaces 68 (one being medially positioned, the other the laterally positioned) is opposite the posterior-chamfer surfaces of the condyles 52, 54. The medial and lateral posterior-chamfer fixation surfaces 68 extend superiorly and posteriorly from their respective medial and lateral distal fixation surfaces 66 in the direction toward their respective posterior fixation surfaces 64. The anterior-chamfer fixation surface 70 is opposite the anterior surface of the femoral component and extends superiorly and anteriorly away from their respective distal fixation surfaces 66 in the direction toward an anterior fixation surface 72. The anterior fixation surface 72 extends generally in the superior/inferior direction.

The bone-engaging surface 62 of the femoral component 12 may also include the outer surfaces of a pair of posts 74 extending superiorly from the distal fixation surfaces 66. The posts 74 are configured to be received into holes formed in the surgically-prepared distal femur of the patient during installation of the femoral component 12.

The femoral component 12 described herein is embodied as a cementless component—that is, the femoral component 12 is designed to be installed on the surgically-prepared distal end of a patient's femur without the use of bone cement. As such, like the tibial tray 16, the bone-engaging surface 62 of the femoral component has the porous-metal coating 32 disposed thereon. Similarly to the tibial tray 16, the porous-metal coating 32 disposed on the femoral component 12 may be a separately-applied coating (e.g., Porocoat® Porous Coating) or the porous-metal coating 32 may disposed on the metallic body 58 of the femoral component 12 by virtue of being additively manufactured contemporaneously with the component's metallic body 58 so as to create a common, monolithic component of the two metal structures. It should be appreciated that although the concepts described herein are particularly useful in the design of cementless components, they may also be utilized in the design of cemented components (i.e., components designed to be implanted with the use of bone cement).

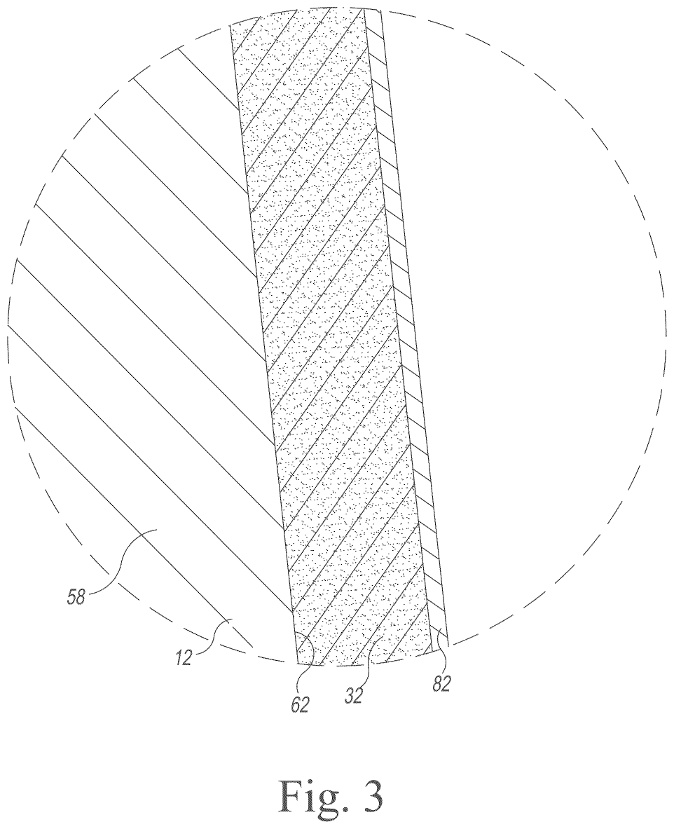

The bone-engaging surface 62 of the femoral component 12 has an antimicrobial layer 82 disposed thereon. As will be discussed below in greater detail, in the case of when the femoral component 12 is constructed with titanium or a titanium alloy, the antimicrobial layer 82 may be a single layer as shown in FIG. 3. This may be achieved by forming the antimicrobial layer 82 directly on the metallic body 58 of the femoral component 12. For instance, the antimicrobial layer 82 may be directly formed on the metallic body 58 without an intermediate structure so that the antimicrobial layer 82 directly contacts the metallic body 58.

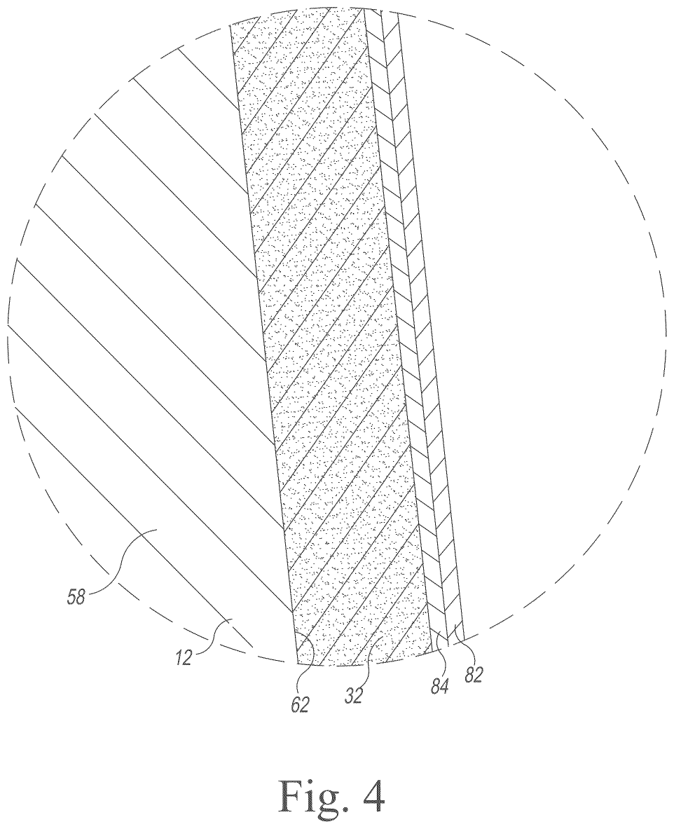

Alternatively, in cases in which the femoral component 12 is constructed of a metal other than titanium or a titanium alloy (e.g., cobalt chromium) a bonding layer 84 may be positioned between the antimicrobial layer 82 and the component's metallic body 58, as shown in FIG. 4. As discussed in greater detail below, the layers 82 and 84 may each be constructed with a material which possesses desirable antimicrobial properties (e.g., capable of reducing or preventing post-surgery infection).

It should be appreciated that, as used herein, the term “layer” is not intended to be limited to a “thickness” of material positioned proximate to another similarly dimensioned “thickness” of material, but rather is intended to include numerous structures, configurations, and constructions of material. For example, the term “layer” may include a portion, region, or other structure of material which is positioned proximate to another portion, region, or structure of differing material. For example, although the interface between the bonding layer 84 and the antimicrobial layer 82 is shown to be uniform in FIG. 4, in some embodiments the interface is irregular such that the bonding layer 84 and the antimicrobial layer 82 do not have a uniform thickness. In some embodiments, a “layer” is formed by modifying a surface or a portion of an existing layer. For example, in some embodiments, the antimicrobial layer 82 is formed from processing at least a portion of bonding layer 84. In alternative embodiments, a “layer” is formed by providing additional material to an existing surface. For example, in some embodiments, the bonding layer 84 is formed by depositing a material onto the metallic body 58 of the femoral component 12, and then further processed to form the antimicrobial layer 82. As will be described in more detail, the bonding layer 84 may be processed completely to become the antimicrobial layer 82. Alternatively, the bonding layer 84 may be partially processed so that a portion becomes the antimicrobial layer 82 and a portion remains the bonding layer 84.

The metallic body 58 of the femoral component 12 may be formed of a metal such as titanium, vanadium, aluminum, cobalt, chromium or a combination thereof. In some embodiments, the metallic body 58 of the femoral component 12 is constructed of a titanium alloy, for example Ti-6Al-4V. When the metallic body 58 of the femoral component 12 is constructed of a metal that does not include titanium, a bonding layer 84 that includes titanium may be deposited on the outer surface 86 of the metallic body 58 of the femoral component 12. For example, the femoral component 12 may include a metallic body 58 constructed of cobalt chromium with a bonding layer 84 that includes titanium or a titanium alloy disposed thereon along its bone-engaging surface 62. The bonding layer 84 may be substantially modified into the antimicrobial layer 82, or a portion of the bonding layer 84 may remain while a portion of the bonding layer 84 is modified into the antimicrobial layer 82. When the metallic body 58 of the femoral component 12 is constructed of titanium, the portion of the metallic body corresponding to the bone-engaging surface 62 itself may be modified to form the antimicrobial layer 82.

The bonding layer 84 may include a titanate. Titanate refers to inorganic compounds that include titanium oxides (TiO₂, TiO, Ti₂O₃, Ti₃O, Ti₂O) for example strontium titanate (SrTiO₃) and titanium anions for example [TiCl₆]²⁻ and [Ti(CO)₇]²⁻. In some embodiments, the titanate includes sodium hydrogen titanate having a formula of NaₓH₂ₓTi_{y}O_{2y}⁺¹ where 0<x<2 and y=2, 3, or 4.

The antimicrobial layer 82 includes a zinc-titanate layer formed from titanate and zinc. Without being limited by theory, it is believed that the zinc ions exchange places with the sodium ions in the titanate to produce the zinc-titanate material. In some embodiments, the antimicrobial layer 82 includes a heterogeneous mixture of zinc hydrogen titanate and sodium hydrogen titanate in a nano-porous form having a formula of ZnₓH₂ₓTi_{y}O_{2y}⁺¹ and NaₓH₂ₓTi_{y}O_{2y}⁺¹ where 0<x<2 and y=2, 3, or 4. As mentioned above, during the process of forming the antimicrobial layer 82, zinc (Zn) replaces sodium (Na) in an ion exchange. In this manner there is a ratio of zinc to sodium (e.g., may be written as Zn₂₋ₓ:Naₓ) within the bone engaging layer so that when one increases, the other decreases. This ratio allows the amount of zinc present in the antimicrobial layer 82 to be adjusted to fit the needs of a given design.

Based on the described ratio (i.e., Zn₂₋ₓ:Naₓ), the antimicrobial layer 82 may have a ratio of zinc to sodium ranging from about 2:0.01 to about 0.01:2 and all possibilities in between. In some embodiments, the ratio of zinc to sodium is about 0.25:1.75, about 0.33:1.66, about 0.5:1.5, about 1:1, or about 1.5:0.5, about 1.66:0.33, or about 1.75:0.25.

The zinc is present in the antimicrobial layer 82 in an amount to provide antimicrobial properties to the femoral component 12. The zinc may be uniformly distributed through the antimicrobial layer 82 in a horizontal and/or a vertical direction to the surface of the femoral component 12. In a uniform distribution example, the concentration of zinc is substantially consistent through the antimicrobial layer 82. Alternatively, the zinc may be non-uniformly distributed through the antimicrobial layer 82 in a horizontal and/or a vertical direction to the surface of the femoral component 12. In a non-uniform distribution example, the concentration of zinc may be higher at an outer surface of the antimicrobial layer 82 and decrease towards the metallic body 58 of the femoral component 12. In another non-uniform distribution example, the concentration of zinc may be higher near the component's metallic body 58 and decrease further out towards the outer surface of the antimicrobial layer 82. When the femoral component 12 includes a bonding layer 84, the concentration of zinc may be higher at its outer surface and decrease towards the bonding layer 84.

Creating a gradient by such varying of the concentration of zinc may allow the length of time and manner in which the femoral component 12 maintains antimicrobial properties. For example, a higher concentration of zinc at its inner surface (i.e., the interface of the antimicrobial layer 82 and the bonding layer 84 or the metallic body 58 (in the absence of a bonding layer 84)) relative to its outer surface may result in the antimicrobial properties remaining or lasting longer (e.g., several days to weeks) relative to if the zinc concentration is higher at the outer surface of the layer 82 (e.g., hours to a few days). However, a higher concentration of zinc at the outer surface of the antimicrobial layer 82 may result in a stronger antimicrobial response at the bony surface upon which the femoral component 12 is installed.

The titanate of the antimicrobial layer 82 may be present at a weight percentage of the total weight of the antimicrobial layer 82 at about 80 wt. % to about 99 wt. %, or about 90 wt. % to about 99 wt. %. The titanate may be present at about 80 wt. % to about 85 wt. %, about 85 wt. % to about 90 wt. %, about 90 wt. % to about 95 wt. %, or about 95 wt. % to about 99 wt. %. In some embodiments, the weight percentage of the titanate contained in the antimicrobial layer 82 is about 88 wt. % to 99 wt. %, about 92 wt. % to about 98 wt. %, about 94 wt. % to about 98%, or about 96 wt. % to about 99 wt. % of the total weight of the antimicrobial layer 82.

The antimicrobial layer 82 may contain zinc as a weight percentage of the total antimicrobial layer 82 weight between about 0.5 wt. % to about 20 wt. %, about 0.5 wt. % to about 15 wt. %, about 0.5 wt. % to about 10 wt. %, about 0.5 wt. % to about 5 wt. %, about 0.5 wt. % to about 1 wt. %, about 1 wt. % to about 20 wt. %, or about 1 wt. % to about 10 wt. %. In some embodiments, zinc is present in the antimicrobial layer 82 as a weight percent of the total antimicrobial layer 82 weight in a range of about 5 wt. % to about 15 wt. %, about 6 wt. % to about 14 wt. %, about 7 wt. % to about 13 wt. %, about 8 wt. % to about 12 wt. %, about 9 wt. % to about 11 wt. %, or about 9 wt. % to about 10 wt. %. In some embodiments, zinc is present in the antimicrobial layer 82 as a weight percent of the total antimicrobial layer 82 weight in a range of about 1 wt. % to about 9 wt. %, about 2 wt. % to about 9 wt. %, about 2 wt. % to about 8 wt. %, about 2 wt. % to about 7 wt. %, about 2 wt. % to about 6 wt. %, or about 2 wt. % to about 5 wt. %. In some embodiments, a portion of the zinc in the antimicrobial layer 82 is available as free zinc.

In some embodiments, the antimicrobial layer 82 has an average thickness of about 10 to about 500 nm, about 10 nm to about 450 nm, about 10 nm to about 400 nm, about 10 nm to about 350 nm, about 10 nm to about 300 nm, about 10 nm to about 250 nm, about 10 nm to about 200 nm, about 10 nm to about 150 nm, about 10 nm to about 100 nm, about 10 nm to 50 nm, about 50 to about 500 nm, about 50 nm to about 450 nm, about 50 nm to about 400 nm, about 50 nm to about 350 nm, about 50 nm to about 300 nm, about 50 nm to about 250 nm, about 50 nm to about 200 nm, about 50 nm to about 150 nm, about 50 nm to 100 nm, about 100 nm to about 500 nm, about 100 nm to about 450 nm, about 100 nm to about 400 nm, about 100 nm to about 350 nm, about 100 nm to about 300 nm, about 100 nm to about 250 nm, about 100 nm to about 200 nm, about 100 nm to about 150 nm, about 150 nm to about 300 nm, about 150 nm to about 250 nm, about 150 nm to about 200 nm, about 200 nm to about 500 nm, about 200 nm to about 450 nm, about 200 nm to about 400 nm, about 200 nm to about 350 nm, about 200 nm to about 300 nm, about 200 nm to about 250 nm, about 250 nm to about 500 nm, about 250 nm to about 450 nm, about 250 nm to about 400 nm, about 250 nm to about 350 nm, or about 250 nm to about 300 nm.

In embodiments of the femoral component 12 that include a bonding layer 84, the bonding layer 84 may have an average thickness of about of about 10 to about 500 nm, about 10 nm to about 450 nm, about 10 nm to about 400 nm, about 10 nm to about 350 nm, about 10 nm to about 300 nm, about 10 nm to about 250 nm, about 10 nm to about 200 nm, about 10 nm to about 150 nm, about 10 nm to about 100 nm, about 10 nm to 50 nm, about 50 to about 500 nm, about 50 nm to about 450 nm, about 50 nm to about 400 nm, about 50 nm to about 350 nm, about 50 nm to about 300 nm, about 50 nm to about 250 nm, about 50 nm to about 200 nm, about 50 nm to about 150 nm, about 50 nm to 100 nm, about 100 nm to about 500 nm, about 100 nm to about 450 nm, about 100 nm to about 400 nm, about 100 nm to about 350 nm, about 100 nm to about 300 nm, about 100 nm to about 250 nm, about 100 nm to about 200 nm, about 100 nm to about 150 nm, about 150 nm to about 300 nm, about 150 nm to about 250 nm, about 150 nm to about 200 nm, about 200 nm to about 500 nm, about 200 nm to about 450 nm, about 200 nm to about 400 nm, about 200 nm to about 350 nm, about 200 nm to about 300 nm, about 200 nm to about 250 nm, about 250 nm to about 500 nm, about 250 nm to about 450 nm, about 250 nm to about 400 nm, about 250 nm to about 350 nm, or about 250 nm to about 300 nm.

It should be appreciated that the antimicrobial layer 82 may further include a hydroxyapatite layer. In some embodiments, the hydroxyapatite layer is disposed on the bone-engaging surface 62 so as to contact the surgically prepared bone.

As alluded to above, when the femoral component 12 is constructed of titanium or a titanium alloy (e.g., Ti-6Al-4V), the antimicrobial layer 82 may be formed integrally with, or otherwise directly on, the bone-engaging surface 62 of the component 12. The method to do so initially includes contacting the bone-engaging surface 62 with a pre-treatment solution to modify the bone-engaging surface 62 to form a titanate-precursor layer. The titanate-precursor layer may include sodium hydroxide titanate. In some embodiments, the titanate precursor layer includes sodium hydrogen titanate in a nano-porous form having a formula of NaₓH₂ₓTi_{y}O_{2y}⁺¹. The precursor solution may include sodium hydroxide (NaOH), for example about 4-7 N concentrated NaOH. The precursor solution may be heated to about 60° C. Further, the precursor solution may be in contact with the femoral component 12 for about 3-5 hours. The temperature and the time may be adjusted as needed depending on the desired thickness and concentration of the titanate-precursor layer to be formed.

A coating solution (the composition of which is described in detail below) then contacts the titanate-precursor layer to produce the antimicrobial layer 82. The titanate-precursor layer is transformed into the antimicrobial layer 82 by the ion exchange of sodium for zinc resulting in the incorporation of zinc into the titanate. The antimicrobial layer 82 includes zinc hydroxide titanate. In some embodiments, the antimicrobial layer 82 includes a mixture of sodium hydroxide titanate and zinc hydroxide titanate. The method may further include removing the femoral component 12 from the coating solution, rinsing the femoral component 12, and/or drying the femoral component 12 after the formation of the antimicrobial layer 82. Further processing steps may be performed on the femoral component 12 before packaging.

In regard to a femoral component 12 constructed of a metal other than titanium or a titanium alloy (e.g., cobalt chromium) or of a metal that does not include a minimal amount of titanium, the above-described method of making the femoral component 12 may include additional steps. In particular, the bonding layer 84 having material that includes titanium is first formed on the metallic body 58 of the femoral component 12. The bonding layer 84 may be pure titanium, a titanium alloy, a titanium oxide or a titanate. For example, the titanium alloy may be Ti-4V-6Al and the titanate may be a titanium oxide such as TiO or TiO₂.

Thereafter, the bonding layer 84 is contacted with the precursor solution in the manner described above to modify some or all of the bonding layer 84 to a titanate-precursor layer. This titanate-precursor layer may then be contacted with the coating solution to form the antimicrobial layer 82 in the manner described above. Depending on the desired outcome, the titanate-precursor layer may be substantially transformed into the antimicrobial layer 82 or a portion of the precursor layer may remain in the final product as the bonding layer 84.

Additionally, the titanate utilized in the method of making the femoral component 12 may include sodium hydroxide titanate. In such an embodiment, the titanate-precursor layer would include sodium hydroxide titanate.

It should be appreciated that certain surfaces (e.g., the articular surface 50) of the femoral component 12 may be masked prior to exposing the component 12 to the precursor and coating solutions. As such, the antimicrobial layer 82 does not cover the total surface area of the femoral component 12. In some embodiments, the antimicrobial layer 82 covers at least about 10%, at least about 20%, at least about 30%, at least about 40%, at least about 50%, at least about 60%, at least about 70%, at least about 80%, or at least about 90% of the femoral component's surface area. In some embodiments, the antimicrobial layer 82 covers about 1% to about 30%, about 1% to about 25%, about 1% to about 20%, about 1% to 15%, about 1% to about 10%, about 1% to about 5%, about 10% to about 90%, about 10%, to about 80%, about 10% to about 70%, about 10% to about 60%, about 10% to about 50%, about 10% to 40%, about 10% to 30%, about 10% to 20% about 20% to about 90%, about 20% to 60%, about 20% to 40%, about 30% to about 70%, about 30% to about 50%, about 45% to about 55%, about 35% to about 65%, or about 10% to about 99% of the femoral component's surface area.

In some embodiments, the coating solution includes a zinc source of about 0.1 mM to about 6 mM, about 0.1 mM to about 5 mM, about 1 mM to about 6 mM, about 1 mM to about 5 mM, about 2 mM to about 5 mM, about 2 mM to about 4 mM, about 1 mM to about 4 mM, about 2 mM to about 6 mM, about 3 mM to about 6 mM, about 3 mM to about 5 mM, or about 3 mM to about 4 mM. In some embodiments, the coating solution comprises a zinc source between about 0.1 mM to about 1 mM, about 1 mM to about 2 mM, about 2 mM to about 3 mM, about 3 mM to about 4 mM, about 3.1 mM to about 4 mM, about 3.2 mM to about 4 mM, about 3.3 mM to about 4 mM, about 3.4 mM to about 4 mM, about 3.5 mM to about 4 mM, about 3.6 mM to about 4 mM, about 3.7 mM to about 4 mM, about 3.8 mM to about 4 mM, or about 3.9 mM to about 4 mM. In some embodiments, the coating solution comprises a zinc source between about 3.4 mM to about 3.8 mM or about 3.5 mM to about 3.7 mM. The amount of zinc present in the coating solution may be adjusted based on the purpose of the zinc.

In some embodiments, the coating solution further includes a phosphate solution. In some embodiments, the phosphate solution includes potassium phosphate (KH₂PO₄) in an amount of about 35 mM to about 45 mM. In some embodiments, the phosphate solution includes KH₂PO₄ at a weight percentage of about 0.01 wt. % to about 0.7 wt. %. In some embodiments, the phosphate solution includes TRIS-(HOCH2)3CNH2 in an amount of about 155 mM to about 160 mM of TRIS-(HOCH2)3CNH2. In some embodiments, the phosphate solution includes TRIS-(HOCH2)3CNH2 at a weight percentage of about 1 wt. % to about 3 wt. %. The phosphate solution may further include sodium chloride (NaCl) in an amount of about 4 M to about 6M. In some embodiments, the phosphate solution includes NaCl at a weight percentage of about 21 wt. % to about 25 wt. %. In some embodiments, the phosphate solution includes water. The phosphate solution may further include water at a weight percentage of about 75 wt. % to about 80 wt. %. In some embodiments, the coating solution has a pH of about 7 to 13. In some embodiments, the coating solution has a pH of about 8 to 12.5. In some embodiments, the coating solution has a pH of about 8 to 10. In some embodiments, the coating solution has a pH of about 10 to 12.5.

In some embodiments, the coating solution further includes sodium hydroxide (NaOH). In some embodiments, the coating solution includes NaOH in an amount of about 2 vol. % to about 3 vol. %. The zinc source, for example zinc nitrate may be present in the coating solution in an amount of about 3 vol. % to about 4 vol. %, and the phosphate solution may be present in an amount of about 92 vol. % to about 95 vol. %.

In some embodiments, the coating solution is substantially free of a phosphate solution. In some embodiments, the coating solution is substantially free of sodium hydroxide (NaOH). In some embodiments, the coating solution is substantially free of a phosphate solution and is substantially free of sodium hydroxide (NaOH). As used herein, the term “substantially free” can refer to a low number or a low concentration, such as less than 5%, less than 4%, less than 3%, less than 2%, less than 1%, less than 0.1% less than 0.01%, and the like.

In some embodiments, the coating solution is devoid of a phosphate solution. In some embodiments, the coating solution is devoid of sodium hydroxide (NaOH). In some embodiments, the coating solution is devoid of a phosphate solution and is devoid of sodium hydroxide (NaOH).

In some embodiments, the coating solution is heated to a temperature between about 60° C. to about 100° C. prior to contacting it with the femoral component 12. The heated temperature of the coating solution is maintained during the step of contacting until such time as the coating solution may be allowed to cool. The duration of time that the solution contacts at the heated temperature may be adjusted depending on the size of the substrate and/or the desired concentration of zinc within the titanate-zinc layer. In some embodiments, the coating solution is heated to a temperature between about 80° C. to about 85° C., about 85° C. to about 90° C., about 90° C. to about 95° C., about 95° C. to about 100° C. The coating solution may be heated to a temperature between 87° C. to about 93° C., or about 90° C.

While the duration of time for contacting at a heated temperature may be adjusted depending on the desired outcomes, the femoral component may be exposed to the coating solution for at least 15 minutes. In some embodiments, the heated coating solution contacts the bone-engaging surface 62 of the femoral component 12 for about 0.5 hours to about 10 hours, about 1 hour to about 10 hours, about 2 hours to about 8 hours, or about 3 hours to about 6 hours.

In some embodiments, heating of the coating solution is performed using a hydrothermal bath to heat the solution within a conductive container. In some embodiments, the process further monitoring the temperature. In some embodiments, the coating solution contacts the entire implant. In some embodiments, the coating solution contacts the bone-engaging surface 62 and does not contact the articular surface 50. For example, masking may be deployed to prevent the coating solution from contacting a portion of the surface of metallic body 58 of the femoral component 12.

In an illustrative method, the heated coating solution is maintained at a temperature of about 90° C. and the femoral component 12 is exposed to it for about 2 to about 4 hours. The result is an antimicrobial layer 82 formed from the surface modification of the titanate precursor layer to the titanate-zinc layer through ion-exchange. As noted above, further processing steps including rinsing, drying, and/or addition of other coatings prior to packaging may be performed. For example, the femoral component may be dried for about one hour at a temperature of about 60° C.

The following Examples are provided to illustrate the method of making the femoral component 12 and its antimicrobial properties.

### EXAMPLES

### Example 1

### Preparation of Coatings on Various Substrates

In the instant example, various types of coated substrates were prepared according to the present disclosure. The exemplary substrates according to the instant example were constructed of a titanium alloy, specifically Ti₆V₄Al. Accordingly, an initial titanium layer (i.e., bonding layer) was not required to be deposited prior to forming the titanate precursor layer.

In the instant example, a hydrothermal pre-treatment was employed to form the titanate precursor layer on each substrate. To do this, the titanium alloy substrates were contacted with 6N NaOH for about 4 hours to produce a titanate precursor layer.

Next, the coating solution was prepared and heated before contacting the titanium alloy samples each having the titanate precursor layer. A hydrothermal bath having a temperature of about 90°-93° C. was employed to heat the coating solution.

In the instant example, the coating solution was prepared using the materials provided in Table 1, with the components of the Phosphate Solution provided in Table 2. The ranges of amounts used for each component are also provided in each table.

TABLE 1
Coating Solution Preparation

| Amount | | Reagent |
|---|---|---|
| 1.5-1.7 | mL | 6N NaOH |
| 2-2.2 | mL | 0.1M Zinc Nitrate |
| 50-60 | mL | Phosphate Solution |
| | | (components provided in Table 2) |

TABLE 2
Phosphate Solution Preparation

| Reagent | Amount per Liter | | Molarity | |
|---|---|---|---|---|
| KH₂PO₄ | 0.272-5.4 | grams | 2-40.00 | mM |
| TRIS-(HOCH₂)₃CNH₂ | 0.9-19.0 | grams | 8-157 | mM |

| | | | |
|---|---|---|---|
| NaCl | 13.6-273.7 | grams | 0.24-4.68M |

| Attribute | Specification Level | — |
|---|---|---|
| pH (Measured @ | 8.32 pH units | — |
| 25° C. +− 0.5° C.) | | |

For the following analyses, the coating solution was prepared using about 1.65 mL 6N NaOH, between 2.0-2.2 mL zinc nitrate (0.1 M), and about 55 mL of phosphate solution of given specific concentrations.

### Example 2

### Antimicrobial Coating Solution Formation

In the instant example, the preferred order of preparing the coating solution was as follows. First, NaOH was added to the phosphate solution. Then, the 0.1 M zinc nitrate was added slowly to the mixture with stirring to allow for the zinc nitrate to fully dissolve. Once complete, the final zinc concentration of the coating solution was between 3.44 mM and 3.78 mM.

The following is an exemplary coating preparation using a hydrothermal bath and the previously described coating solution to treat multiple samples.

Covered glass beakers with the coating solution were placed in a hydrothermal bath to warm the coating solution gradually to and maintain at about 90° C. The glass beakers (i.e., reaction vessels) were covered to minimize evaporation and reduce solution concentration variations. Each sample was placed into its own reaction vessel to contact the coating solution and undergo a reaction (i.e., form an antimicrobial layer on the surface of the substrate). Each sample was allowed to contact the heated coating solution for about 2-4 hours.

In the instant example, the sample was prepared without the use of motion. Alternatively, the samples may be slightly agitated during preparation (e.g., continuously or periodically) or not agitated at all.

### Example 3

### Rinsing of Coated Samples

After forming the antimicrobial layer, the samples were removed from the coating solution and rinsed. In the instant example, the samples are rinsed three times with de-ionized (DI) water in a beaker for about 30 seconds, each. Next, the coated samples were allowed to dry. The coated samples are air dried or dried in an oven in order to remove moisture.

After coating, the amount of zinc on the samples varied from 1% to 2.5% as detected by an energy dispersive spectroscopy.

### Example 4

### Antimicrobial Evaluation

The antimicrobial layers having a titanate-zinc layer can be evaluated for their antimicrobial properties. For instance, samples can be tested using bacterial cells to analyze antimicrobial effects.

In the instant example, bacterial cells were warmed in a table-top shaker for about hour before use. Prior to contacting the samples with the bacteria, the samples were sterilized using known methods (e.g., placing the samples under a UV light for about 20 minutes before use).

Media Assay: To evaluate the antimicrobial properties, each sample was contacted with a growth solution containing bacteria. In an exemplary analysis, 5 mL of a growth solution comprising 2% sterile tryptic soy broth (TSB) was prepared. The growth solution was prepared by combining 100 μL of TSB to 4.89 mL of DI water in a container (for example, Thermo Scientific, 2118-0002 60 mL). Then, 10 μL of a Staphylococcus aureus (SA) inoculum was added to the growth solution to result in a total volume of 5 mL. Once the solution was prepared in each container, a single sample was placed inside each container to contact the growth solution. The containers were sealed, for example with a lid, and allowed to incubate with some movement, for example rocking or shaking, for 24±2 hours.

After incubation was completed, serial dilutions of the growth media were prepared. Performing serial dilutions is a common practice in cell biology. In the instant example, amounts of 10² and 10⁶-10⁸ dilutions were plated onto CompactDry X-SA growth plates (Hardy Diagnostics). After 36-48 hours, the growth plates were examined and colonies were counted. The plates are shown in FIG. 7.

Surface Colonization Assay: Each sample was rinsed three times with autoclaved DI water (e.g., about 10 mL of DI water was used each time for a total of about 30 mL). Next, each washed coated sample was placed into its own clean, sterile vessel.

Thereafter, 6 mL of growth media was added to each sterile vessel that contained a coated sample. For the instant example, the growth media was prepared by adding 120 μL of TSB to 5.88 mL of DI water. The vessels were then closed/sealed and sonicated for about 1 minute. This allowed the growth media to interact with its respective sample.

After sonication, 5 mL of the growth solution was transferred to a new container (e.g., a capped glass test tube (Becton Dickinson, 352057 Falcon) and placed into a table-shaker incubator and cultured for 24±2 hours. After culturing, serial dilutions and plating of 10² and 10⁶-10⁸ dilutions were performed as previously described in order to analyze whether bacteria were able to reproduce and grow on the titanate-zinc layer of each sample.

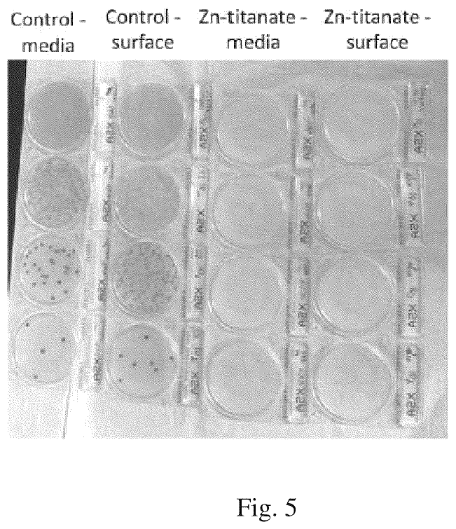

After 36-48 hours, the growth plates were examined and colonies were counted. The plates are shown in FIG. 5.

The microbial evaluations indicated that the samples having a titanate-zinc layer reduced bacterial growth by at least two orders of magnitude (by about 99%).

### Example 5

### Antimicrobial Efficacy Evaluation

Elements of the present disclosure can also be evaluated using other mechanism. The analysis of the instant example provides measurement of antibacterial activity on plastic surfaces and is based on ISO 22196.

For the instant example, four different groups of plastic sample dishes were made: i) a first control group evaluating antimicrobial efficacy in media; ii) a second control group evaluating the titanate surface; iii) a group having zinc and titanate evaluating antimicrobial efficacy in media, and iv) a group having zinc evaluating the Zn-titanate (3-4%) surface.

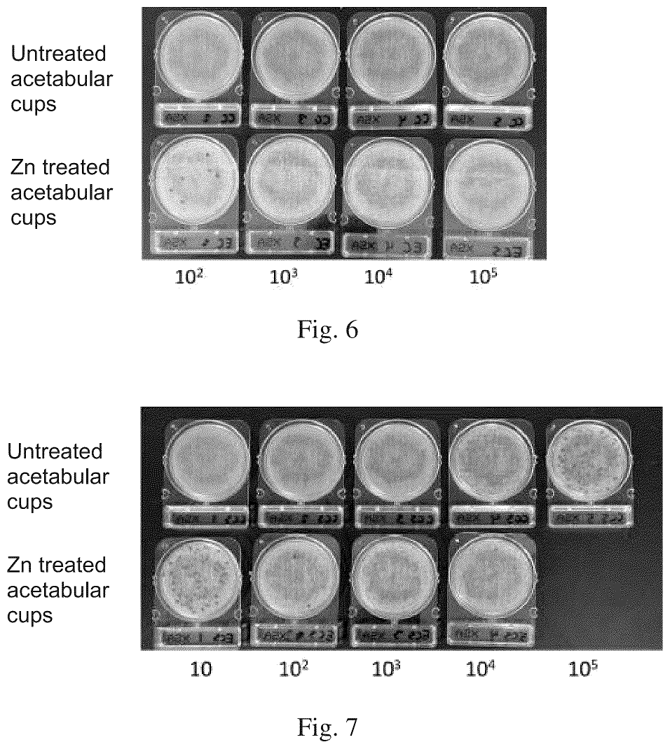

The four groups were then tested for antibacterial properties by ascertaining planktonic time-kill and surface viability after being inoculated with 106 colony forming units (CFU) of SA. As shown in FIGS. 6 and 7, the third and fourth groups (i.e., those including zinc) demonstrated efficacy in their antibacterial effects. Specifically, a 4-log efficacy against SA was observed for the zinc-including groups, suggesting that inclusion of zinc provides for killing of bacteria on contact as well as at a distance. As shown in FIGS. 6 and 7, the top row of untreated acetabular cups contained too many SA colonies to count.

While the disclosure has been illustrated and described in detail in the drawings and foregoing description, such an illustration and description is to be considered as exemplary and not restrictive in character, it being understood that only illustrative embodiments have been shown and described and that all changes and modifications that come within the spirit of the disclosure are desired to be protected.

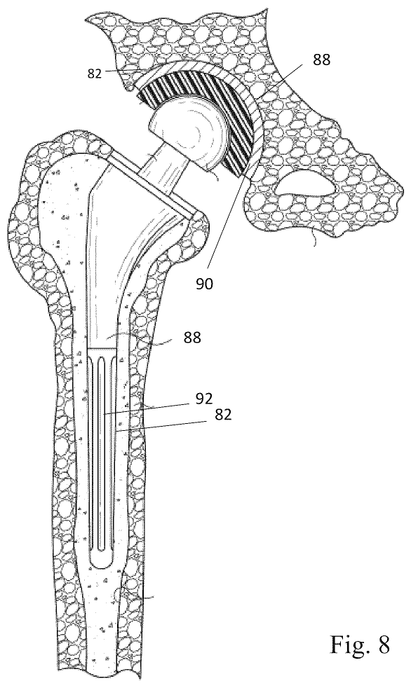

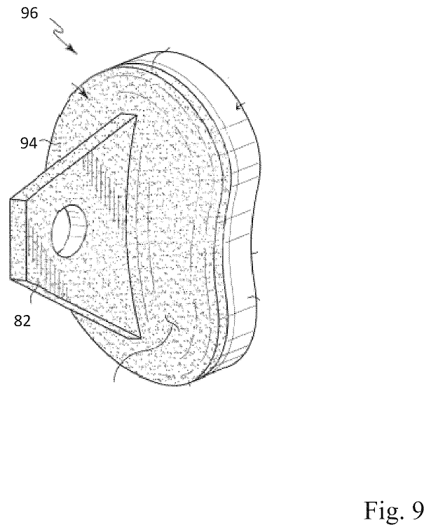

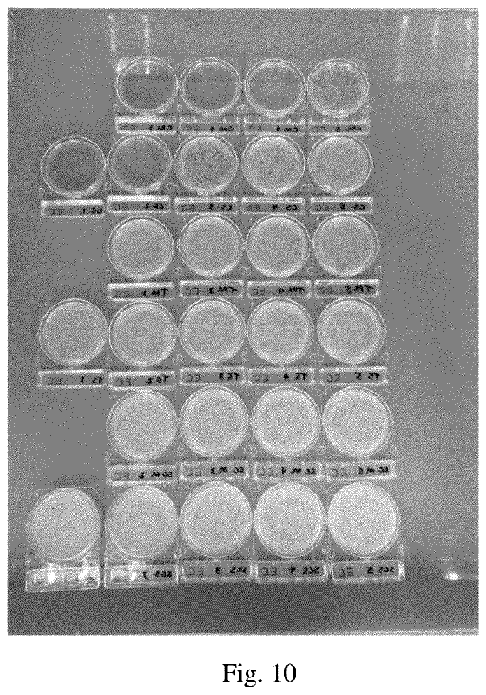

For example, although the concepts of the present disclosure have been discussed in detail in regard to forming the antimicrobial layer 82 on the femoral component 12, it should be appreciated that the antimicrobial layer 82 may be formed on other types of orthopaedic implants. For example, the antimicrobial layer 82 may be formed on the bone-engaging surface 28 of the tibial tray 16. Moreover, as shown in FIG. 8 the antimicrobial layer 82 may be formed on the bone-engaging surfaces 88 of acetabular cup 90 and femoral stem 92 of a hip prosthesis. Likewise, as shown in FIG. 9, the antimicrobial layer 82 may be formed on the bone-engaging surface 94 of a glenoid component 96 and humeral component (not shown) of a shoulder prosthesis. In essence, the antimicrobial layer 82 may be formed on the bone-engaging surfaces of any implantable prosthesis.

### Example 6

### Antimicrobial Efficacy Evaluation

Elements of the present disclosure can also be evaluated using other mechanisms. The analysis of the instant example provides measurement of antibacterial activity on various samples.

For the instant example, 100 μl of E. coli bacteria culture was added to 2.5 ml of tryptic soy broth (TSB), then placed in a tabletop shaker incubator for 24 hours. Thereafter, on Day 1, dilutions containing 1×10³ of E. coli inoculum and a 2% TSB solution were prepared. The following samples were prepared using the noted amounts in Tables 3-5:

TABLE 3
Sample 1 - Control (untreated tibial tray; solution deposited
hydroxyapatite (SoDHA) treated)

| | | |
|---|---|---|
| DI water | 88.19 | ml |
| TSB | 1.8 | ml |
| Inoculum | 10 | μl |
| Total solution volume | 90 | ml |

TABLE 4
Sample 2 - Zinc treated tibial tray (SoDHA treated)

| | | |
|---|---|---|
| DI water | 88.19 | ml |
| TSB | 1.8 | ml |
| Inoculum | 10 | μl |
| Total solution volume | 90 | ml |

TABLE 5
Sample 3 - Zinc treated coupon (solution deposited hydroxyapatite
(SoDHA) treated)

| | | |
|---|---|---|
| DI water | 5.87 | ml |
| TSB | 120 | μl |
| Inoculum | 10 | μl |
| Total solution volume | 6 | ml |

On Day 2, the designated amount of serial dilutions of the media and plates was performed for all solutions onto CompactDry EC plates. Approximately 900 uL of each dilution was added to the center of each CompactDry EC plate, pipetting out slowly. Liquid was then emptied from the containers using an aspirator and the designated amount of water was added to each container. The sample was shaken back and forth five times. The liquid emptying, water addition, and shaking was repeated three times.

A new container was provided for each sample and the designated amount of fresh DI water was added for each sample volume. The samples were moved into the new containers and sonicated for 1 minute with a closed lid.

After sonicating, the designated dilutions of the solution was performed to represent the surface activity and plate onto CompactDry EC plates. The plates were placed in the incubator and checked after 24 hours.

FIG. 10 shows the bacterial counts of the various samples. As shown in FIG. 10, the surfaces treated with zinc demonstrated little to no bacterial growth on either the tibial tray or the coupon. However, bacterial growth was shown on the control samples, indicating that the inoculation step was successfully performed.

### Example 7

### Comparison of Zinc Nitrate Treatment Solutions

The instant example provides analysis of various treatment solutions comprising zinc on titanium surfaces. For instance, titanium coupons were treated with different hydrothermal treatment processes as described below.

For Group 1, titanium coupons were oven etched and treated with a ‘general’ preparation as follows. First, a stock solution comprising approximately 2.4 mM of phosphate was prepared. Second, a solution comprising NaOH and zinc nitrate was prepared. The concentration ratios of phosphate, NaOH, and zinc nitrate for the Group 1 preparations were as follows in Table 6:

TABLE 6

| Concentration Ratios | Ratio |
|---|---|
| Phosphate/Zinc Nitrate | 1.0657 |
| NaOH/Phosphate | 42.4555 |
| NaOH/Zinc Nitrate | 45.2434 |

The stock solution and the NaOH/zinc nitrate solution were combined and titanium coupons were placed into the combined treatment solution. After 4 hours, the titanium coupons were rinsed with DI water and sonicated. The titanium coupons were then placed into an oven to dry before analysis was performed.

For Group 2, titanium coupons were oven etched and treated with an increased zinc nitrate solution as follows. First, a stock solution comprising approximately 2.4 mM of phosphate was prepared. Second, a solution comprising NaOH and approximately 25% increased zinc nitrate was prepared. The concentration ratios of phosphate, NaOH, and zinc nitrate for the Group 2 preparations were as follows in Table 7:

TABLE 7

| Concentration Ratios | Ratio |
|---|---|
| Phosphate/Zinc Nitrate | 0.8478 |
| NaOH/Phosphate | 42.6905 |
| NaOH/Zinc Nitrate | 36.1947 |

The stock solution and the NaOH/zinc nitrate solution were combined and titanium coupons were placed into the combined treatment solution. After 4 hours, the titanium coupons were rinsed with DI water and sonicated. The titanium coupons were then placed into an oven to dry before analysis was performed.

For Group 3, titanium coupons were oven etched and treated with a zinc nitrate solution only. The titanium coupons were treated without use of phosphate or NaOH. The titanium coupons were placed into the zinc nitrate-only solution. After 4 hours of treatment, the titanium coupons were rinsed with DI water and sonicated. The titanium coupons were then placed into an oven to dry before analysis was performed.

Surprisingly, the titanium coupons that were treated with a zinc nitrate solution only (i.e., treated without use of phosphate or NaOH) demonstrated the highest percentage of zinc on the titanium structures. Table 8 shows the comparison of zinc percentages in the titanium samples:

TABLE 8

| Sample | Group 1 | Group 2 | Group 3 |
|---|---|---|---|
| Zinc (ZN) % | 3.59 | 4.53 | 10.51 |
| | 4.09 | 4.92 | 9.91 |
| | 3.87 | 4.07 | 9.04 |
| | 3.64 | 4.59 | 9.48 |
| Average Zn % | 3.80 | 4.53 | 9.74 |

Thus, the titanium samples treated with a zinc nitrate solution only (i.e., treated without use of phosphate or NaOH) provided the highest zinc percentages and greatest flexibility in the instant example. These results were unexpected.

There are a plurality of advantages of the present disclosure arising from the various features of the method, apparatus, and system described herein. It will be noted that alternative embodiments of the method, apparatus, and system of the present disclosure may not include all of the features described yet still benefit from at least some of the advantages of such features. Those of ordinary skill in the art may readily devise their own implementations of the method, apparatus, and system that incorporate one or more of the features of the present invention and fall within the spirit and scope of the present disclosure as defined by the appended claims.

1. An orthopaedic knee prosthesis comprising:
an implantable orthopaedic component, the orthopaedic component having a bone-engaging surface with an antimicrobial layer disposed thereon,
wherein the antimicrobial layer comprises zinc hydroxide titanate in a nano-porous form.

2. The prosthesis of claim 1, wherein the zinc is present in an amount to provide antimicrobial properties to the orthopaedic component.

3. The prosthesis of claim 1, wherein the zinc is uniformly distributed through the antimicrobial layer.

4. The prosthesis of claim 1 further comprising a bonding layer positioned between the bone-engaging surface and the antimicrobial layer.

5. The prosthesis of claim 4, wherein the bonding layer comprises titanate.

6. The prosthesis of claim 4, wherein the bonding layer comprises a first portion of titanium and a second portion of titanate.

7. The prosthesis of claim 6, wherein the first portion of titanium comprises a titanium material selected from pure titanium, a titanium alloy, or a titanium oxide.

8. The prosthesis of claim 5, wherein the titanate comprises sodium hydroxide titanate.

9. The prosthesis of claim 1, wherein the antimicrobial layer comprises a ratio of zinc to sodium (Zn₂₋ₓ:Naₓ) ranging from about 2:0 to about 0:2.

10. The prosthesis of claim 1, wherein the bone-engaging surface of the orthopaedic component comprises titanium.

11. The prosthesis of claim 1, wherein the bone-engaging surface of the orthopaedic component is devoid of titanium.

12. The prosthesis of claim 11, wherein the bone-engaging surface of the orthopaedic component comprises cobalt chromium.

13. The prosthesis of claim 1, wherein the antimicrobial layer is at a weight percentage between about 0.001 wt. % to about 10 wt. % of the total weight of the orthopaedic component.

14. The prosthesis of claim 1, wherein the antimicrobial layer comprises zinc at a weight percentage of the total weight of the antimicrobial layer between about 1 wt. % to about 50 wt. %, about 1 wt. % to about 40 wt. %, about 1 wt. % to about 30 wt. %, about 1 wt. % to about 20 wt. %, or about 1 wt. % to about 10 wt. %.

15. A method of making an implantable orthopaedic component having antimicrobial properties, comprising:
contacting a bone-engaging surface of the orthopaedic component with a precursor solution to form a precursor layer, wherein the precursor solution comprises sodium hydroxide, and
contacting the precursor layer with a coating solution comprising a source of zinc so as to allow an ion exchange between the precursor layer and the source of zinc to produce an antimicrobial layer.

16. The method of claim 15, wherein the precursor layer comprises titanate.

17. The method of claim 15, wherein the coating solution further comprises a phosphate solution and sodium hydroxide.

18. The method of claim 17, wherein the coating solution is substantially free of a phosphate solution.

19. The method of claim 17, wherein the coating solution is substantially free of sodium hydroxide.

20. The method of claim 17, wherein the coating solution is substantially free of a phosphate solution and wherein the coating solution is substantially free of sodium hydroxide.
